(19) United States
(12) Patent Application Publication
(10) Pub. No.: US 2017/0319374 A1
(43) Pub. Date: Nov. 9, 2017

(54) AUTOMATIC FLUID DRAINAGE DEVICE

(71) Applicant: Shaoqin Hu, Guangzhou City (CN)

(72) Inventor: Shaoqin Hu, Guangzhou City (CN)

(21) Appl. No.: 15/527,625

(22) PCT Filed: Nov. 11, 2015
(86) PCT No.: PCT/CN2015/094354

§ 371 Date: May 17, 2017

(30) Foreign Application Priority Data
Nov. 24, 2014 (CN) 201410679136.X
Feb. 22, 2015 (CN) 201510085893.9

(51) Int. Cl. A61F 5/44; A61J 1/20; A61M 1/00; A61F 5/453; A61F 5/455

(52) U.S. Cl. CPC A61F 5/4407; A61J 1/2037; A61M 1/0009; A61M 1/0029; A61F 5/455; A61F 5/453

(57) ABSTRACT

An automatic drainage device comprises a drainage container separated by a partition plate into a fluid storage cavity provided with a connecting pipe and a fluid discharge channel provided with a fluid discharge outlet. The partition plate is provided with a fluid drainage outlet that connects the fluid storage cavity to the fluid discharge channel. The device further comprises a movable gasket disposed below the partition plate for closing the fluid drainage outlet, and a control device comprising a remote control receiving module or a control device configured to monitor a liquid level in the fluid storage cavity, to move the movable gasket to open or close the fluid drainage outlet. The automatic drainage device remotely activates the control device by a remote control receiving module or by monitoring the status of the liquid level, to control the opening and closing of the fluid drainage outlet.

### TECHNICAL FIELD

The present invention relates to the field of medical instruments, and more particularly, to an automatic drainage device.

### BACKGROUND

In clinical care, nursing personnel needs to regularly check a drainage device for storing the drainage fluid of patients, record the reserves of the drainage fluid and drain the drainage fluid without delay, so as to avoid spilling or flowing back to the human body to cause iatrogenic pollution after the drainage fluid is filled.

Clinically, the fluid volume is fundamentally used as a recording basis for the treatment of drainage fluid. A mechanical automatic catheterization bag has the status of low precision. There is an automatic catheterization system that applies an electronic mode, and mostly uses the hydraulic pressure and weight as the detection basis, but when the urine proportion of the patient is different, the volume is conversed with a certain error, and such products give priority to solving intravesical pressure monitoring and bladder function training of the patients, but cannot solve the heavy, dirty and infectious work of dumping urine excrement in catheterization care, and cannot reduce the working pressure and the risk of infection of the nursing personnel.

At the same time, the automatic catheterization system in the electronic mode needs to place part of the electronic components in a drainage catheter. If an external insulation layer is damaged, the drainage fluid is prone to be charged, thus endangering the security of patients.

When an electronic fluid discharge valve of the current electronic catheterization product is subjected to power failure or other failure, there is no backup solution, which easily leads to the backflow of the drainage fluid, resulting in medical malpractices.

During the care of the patients who are suffered from the Ebola, plague and other major infectious diseases, it will increase the risk of infection of the nursing personnel if the nursing personnel deals with the urine excrement with hands, so that a product that has high security and can avoid a direct contact is needed.

### SUMMARY

In order to solve the above-mentioned problem, the present invention provides an automatic drainage device with high precision and excellent safety.

An automatic drainage device comprises: a drainage container; a partition plate, by which the drainage container is separated into a fluid storage cavity located at an upper portion thereof and a fluid discharge channel located at a lower portion thereof, wherein the fluid storage cavity is provided with a connecting pipe, the fluid discharge channel is provided with a fluid discharge outlet, the partition plate is provided with a fluid drainage outlet for connecting the fluid storage cavity and the fluid discharge channel; a movable gasket, for closing the fluid drainage outlet; and a control device, comprising a remote control receiving module and configured to controllably force the movable gasket to move for opening the fluid drainage outlet.

Another automatic drainage device comprises: a drainage container; a partition plate, by which the drainage container is separated into a fluid storage cavity located at an upper portion thereof and a fluid discharge channel located at a lower portion thereof, wherein the fluid storage cavity is provided with a connecting pipe, the fluid discharge channel is provided with a fluid discharge outlet, the partition plate is provided with a fluid drainage outlet for connecting the fluid storage cavity and the fluid discharge channel; a movable gasket, for closing the fluid drainage outlet; and a control device, configured to monitor a liquid level in the fluid storage cavity and thereby to controllably force the movable gasket to move for opening and/or closing the fluid drainage outlet.

As an improvement of the technical solution of the present invention, the automatic drainage device further comprises an electromagnetic device for closing the fluid drainage outlet by means of the movable gasket, wherein the control device comprises at least one liquid level monitoring unit comprising a fixed electrode and a movable electrode, while the liquid level rises to a drainage level, the movable electrode moves into contact with the fixed electrode and a circuit is switched on to generate a signal upon which the control device opens the fluid drainage outlet by means of the movable gasket.

As an improvement of the technical solution of the present invention, the partition plate and the movable gasket are provided with an electromagnetic device and a magnetic iron sheet in a built-in manner, respectively, which are and mutually attracted for closing the fluid drainage outlet, and the electromagnetic device is disposed in the partition plate or the movable gasket.

As an improvement of the technical solution of the present invention, the liquid level monitoring unit further comprises a suspension iron sheet which is fixed by one end thereof, and the movable electrode is fixed at a top end of the suspension iron sheet.

As an improvement of the technical solution of the present invention, the control device is disposed on a side wall of the drainage container, and the control device comprises a liquid level monitoring unit that is flush with a bottom of the fluid storage cavity, and at least one liquid level monitoring unit corresponding to a drainage height.

As an improvement of the technical solution of the present invention, the drainage container is further provided with a vertically-disposed buoy motion rod, and a buoy that can slide up and down along with the buoy motion rod following the liquid level therein, and the buoy is provided with a magnetic material.

As an improvement of the technical solution of the present invention, the control device is disposed at the bottom of the partition plate on one side, the partition plate is provided with a liquid level monitoring opening, an elastic closing film and a liquid level monitoring unit which are disposed below the liquid level monitoring opening, the control device comprises at least two fixed electrodes which are vertically disposed in parallel below the elastic closing film, the movable electrode on the elastic closing film contacts with the fixed electrode located above when the liquid level equals to zero, and the movable electrode on the elastic closing film contacts with the fixed electrode located below when the liquid level is maximum.

As an improvement of the technical solution of the present invention, the movable gasket is by one end thereof hingedly mounted in the fluid discharge channel, and the device further comprises a limiting stopper disposed below a movable end of the movable gasket.

As an improvement of the technical solution of the present invention, the control device further comprises an energy module and a data processing center, and the energy module is a built-in power source and/or an external power source interface, the control device further comprises a space position judger for determining a placement status of the drainage container.

As an improvement of the technical solution of the present invention, the control device further comprises an alarm apparatus for indicating a fluid level in the fluid storage cavity.

As an improvement of the technical solution of the present invention, the control device further comprises a remote control receiving module, and is configured to forcedly open the fluid drainage outlet by means of the movable gasket.

As an improvement of the technical solution of the present invention, the remote control receiving module controls at least the drainage container to conduct three procedures including forced discharge, locking fluid and automatic drainage.

As an improvement of the technical solution of the present invention, the automatic drainage device further comprises a data display screen on the drainage container, for displaying data of drainage time, total drainage volume and drainage duration of the drainage container.

As an improvement of the technical solution of the present invention, the fluid drainage outlet is automatically opened when the fluid in the fluid storage cavity is excessive.

The present invention has the beneficial effects as follows: the automatic fluid discharge and drainage device remotely triggers the control device by using a remote control contact module or activates the control device by monitoring the status of the liquid level, to control movement of the movable gasket, so as to indirectly control the fluid drainage outlet to be opened and closed. The fluid discharge and drainage device has higher precision, requires no manual operation in the fluid discharge process, can accurately record the fluid drainage status, effectively reduces labor intensity of nursing personnel and prevents an operator from being inflected, and has better security and convenience.

### BRIEF DESCRIPTION OF THE DRAWINGS

The present invention is further illustrated with reference to drawings thereinafter.

FIG. 1 is a schematic view of an automatic drainage device in a fluid storage status according to a first embodiment of the present invention;

FIG. 2 is a schematic view of the automatic drainage device in a fluid drainage status according to the first embodiment of the present invention;

FIG. 3 is a schematic view of an automatic drainage device in a fluid storage status according to a second embodiment of the present invention;

FIG. 4 is a schematic view of an automatic drainage device in a fluid drainage status according to the second embodiment of the present invention;

FIG. 5 is a schematic view of an automatic drainage device which is fully assembled, according to an embodiment of the present invention;

FIG. 6 is a schematic view of a housing according to an embodiment of the present invention.

### DETAILED DESCRIPTION

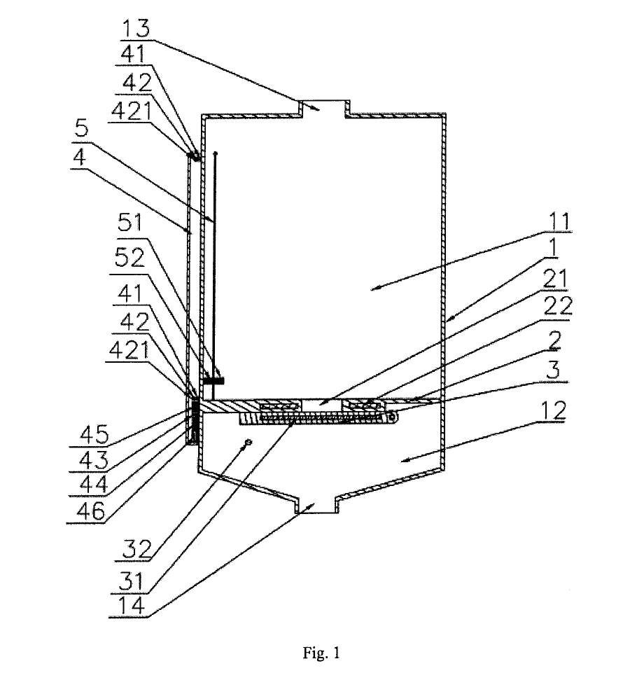

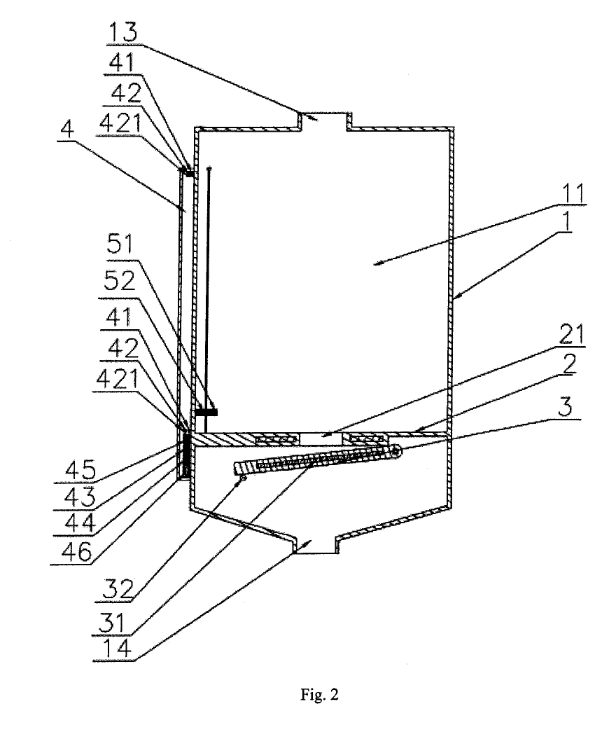

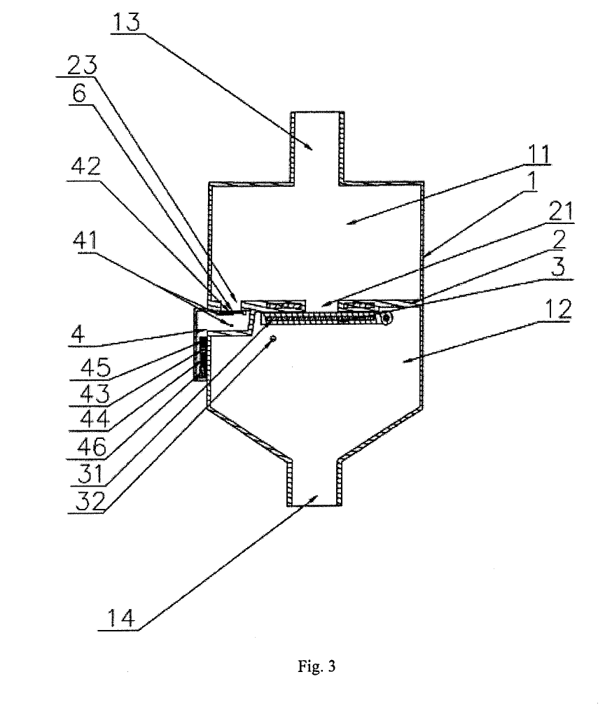

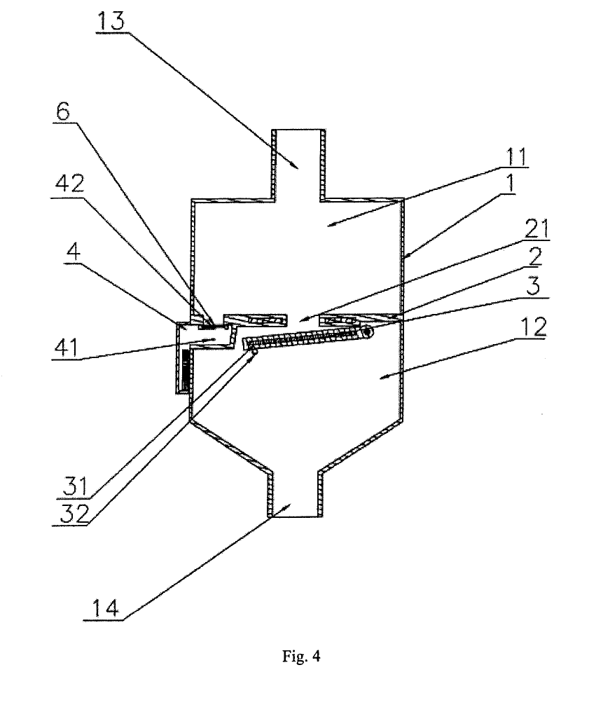

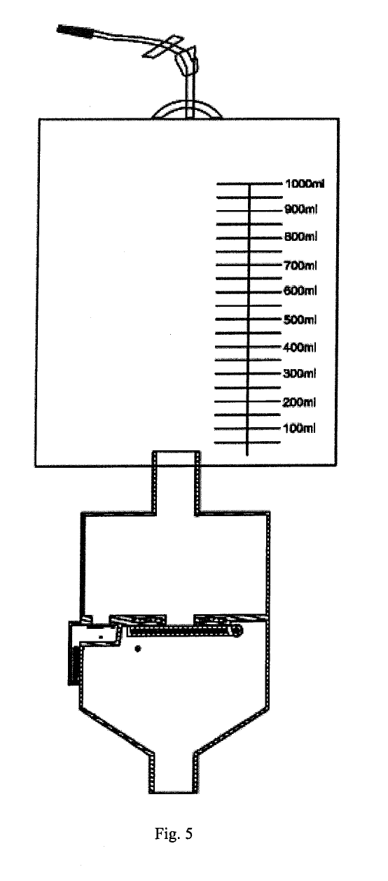

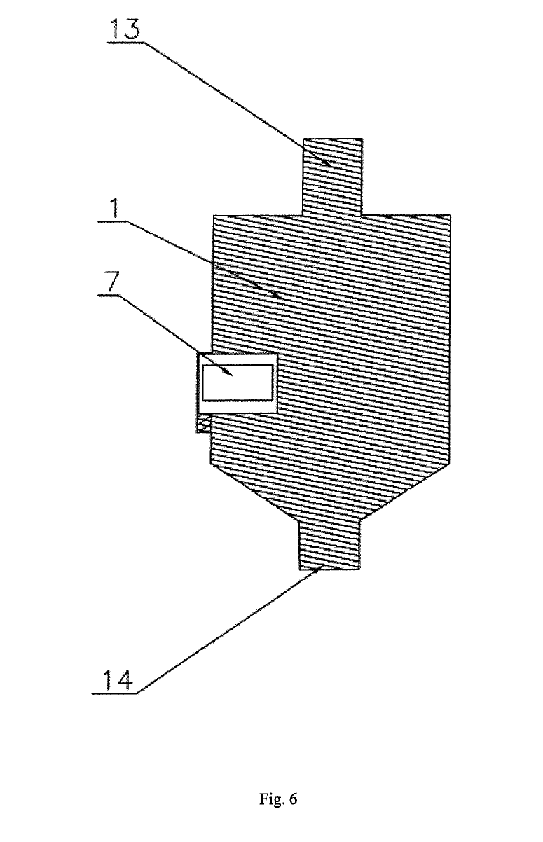

As shown in FIGS. 1 to 6, an automatic drainage device comprises: a drainage container 1; a partition plate 2, by which the drainage container 1 is separated into a fluid storage cavity 11 located at an upper portion thereof and a fluid discharge channel 12 located at a lower portion thereof, wherein the fluid storage cavity 11 is provided with a connecting pipe 13, the fluid discharge channel 12 is provided with a fluid discharge outlet 14, the partition plate 2 is provided with a fluid drainage outlet 21 for connecting the fluid storage cavity 11 and the fluid discharge channel 12; a movable gasket 3, for closing the fluid drainage outlet 21; a control device 4, comprising a remote control receiving module and configured to controllably force the movable gasket 3 to move for opening the fluid drainage outlet 21.

According to another preferred embodiment of the invention, an automatic drainage device comprises: a partition plate 2, by which the drainage container 1 is separated into a fluid storage cavity 11 located at an upper portion thereof and a fluid discharge channel 12 located at a lower portion thereof, wherein the fluid storage cavity 11 is provided with a connecting pipe 13, the fluid discharge channel 12 is provided with a fluid discharge outlet 14, the partition plate 2 is provided with a fluid drainage outlet 21 for connecting the fluid storage cavity 11 and the fluid discharge channel 12; a movable gasket 3, for closing the fluid drainage outlet 21; a control device 4, configured to monitor a liquid level in the fluid storage cavity 11 and thereby to controllably force the movable gasket 3 to move for opening and/or closing the fluid drainage outlet 21.

The automatic drainage device remotely activates the control device by means of a remote control contact module, or by monitoring the status of the liquid level, for controlling the movement of the movable gasket 3, so as to indirectly control the opening/closing of the fluid drainage outlet 21. Resulting from above, the automatic device has a higher precision, requires no manual operation in its fluid discharge process, can accurately record the fluid drainage status, effectively reduces labor intensity of nursing personnel and prevents an operator from being inflected, and has better security and convenience.

Moreover, according to the invention, electric leakage accident is avoided as external circuits and electronic elements are adopted in the automatic drainage device.

Moreover, according to the invention, a failure settle scheme of configuring the fluid drainage outlet 21 to be automatically opened in case which the fluid volume is excessive in the fluid storage cavity 11, is adopted to avoid retrograde infection and other medical malpractices.

In a preferred embodiment of the present invention, the movable gasket 3 is arranged below the partition plate 2, and configured to automatically open the fluid drainage outlet 21 when the fluid volume in the fluid storage cavity 11 is excessive. The movable gasket 3 and the partition plate 2 are closed by an electromagnetic device. The control device 4 comprises at least one liquid level monitoring unit comprising a fixed electrode 41 and a movable electrode 42, the movable electrode 42 is operative to move into contact with the fixed electrode 41 to activate a circuit when the liquid level rises to a fluid drainage liquid level, so as to generate a signal to open the movable gasket 3 through the control device 4.

The movable gasket 3 is preferably a cuboid, the movable gasket 3 may be hinged in the fluid storage cavity 11 as long as the electromagnetic device is arranged above the movable gasket 3. Alternatively, the movable gasket 3 maybe a piston or in other shapes. Similarly, the movable gasket 3 can be arranged at any position about the fluid drainage outlet.

In the automatic drainage device, two separated electrodes, including a fixed electrode 41 and a movable electrode 42 which are moved into contact with each other to generates an electrical signal based upon the fluid level, are used to control the movement of the movable gasket 3, thereby to indirectly control the opening/closing of the fluid drainage outlet 21. The fluid drainage of the drainage container 1 is conducted upon the electronic signal derived from the detection on the fluid level, such that the automatic drainage device has higher precision, requires no manual operation in the fluid discharge process, can prevent an operator from being inflected, and has better security.

In a preferred embodiment of the present invention, the partition plate 2 and the movable gasket 3 are mutually attracted by means of an electromagnetic device 22 and a magnetic iron sheet 31 respectively built therein, to close the fluid drainage outlet 21, and the electromagnetic device 22 is disposed in the partition plate 2 or the movable gasket 3.

In another preferred embodiment of the present invention, the fluid drainage outlet 21 is automatically opened when the fluid in the fluid storage cavity 11 is excessive in capacity. Specifically, when the fluid in the fluid storage cavity 11 is excessive, the fluid pressure in the fluid storage cavity 11 is greater than the attractive force between the electromagnetic device 22 and the magnetic iron sheet 31, therefore the movable gasket 3 automatically is opened.

The electromagnetic device 22 and the magnetic iron sheet 31 are configured to have a normal drainage function, as well as an emergency drainage function in an emergency, without an additional emergency drainage outlet, so as to have a lower manufacturing cost. Moreover, this ensures that the drainage fluid will not flow back when the automatic drainage device is broken down, so as to avoid medical malpractices that may cause damage to the health of the patients.

In a preferred embodiment of the present invention, the liquid level monitoring unit further comprises a suspension iron sheet 421 which is fixed by one end thereof, and the movable electrode 42 is fixed at a top end of the suspension iron sheet 421.

In a preferred embodiment of the present invention, the control device 4 is disposed on a side wall of the drainage container 1, and the control device 4 comprises a liquid level monitoring unit that is flush with the bottom of the fluid storage cavity 11 and at least one liquid level monitoring unit that is corresponding to a drainage height.

In a preferred embodiment of the present invention, the drainage container 1 is further provided with a vertically-disposed buoy motion rod 5, and a buoy 51 that can slide up and down along with the buoy motion rod 5 following the liquid level, and the buoy 51 is provided with a magnetic material 52.

In a preferred embodiment of the present invention, the control device 4 is disposed at the bottom of one side of the partition plate 2, the partition plate 2 is provided with a liquid level monitoring opening 23, an elastic closing film 6 and a liquid level monitoring unit. The elastic closing film 6 and the liquid level monitoring unit are disposed below the liquid level monitoring opening 23. The control device 4 further comprises at least two fixed electrodes 41 that are vertically disposed in parallel below the elastic closing film 6, the movable electrode 42 on the elastic closing film 6 contacts with the fixed electrode 41 located above when the liquid level equals to zero, and the movable electrode 42 on the elastic closing film 6 contacts with the fixed electrode 41 located below when the liquid level is maximum. The maximum of the final liquid level is specifically set according to actual conditions.

In a preferred embodiment of the present invention, one end of the movable gasket 3 is hingedly mounted in the fluid discharge channel 12, and the movable gasket 3 is further provided with a limiting stopper 32 disposed below a movable end of the movable gasket 3.

The control device 4 can be realized in two modes, in which the structure of the liquid monitoring unit remains unchanged.

In a first mode of the control device 4, the fixed electrode 41 is located between the movable electrode 42 and the buoy motion rod 5, the bottom of the movable electrode 42 is fixed below the suspension iron sheet 421, and the buoy 51 drives the magnetic material 52 disposed in the buoy 51 to move up or down following the liquid level. When the buoy 51 rises to close to the set fluid drainage position, the magnetic material 52 pulls the movable electrode 42 to move toward the position where the fixed electrode 41 is located via magnetic attractive force. When the buoy 51 is finally located in the set fluid drainage position, the movable electrode 42 contacts with the fixed electrode 41, so that the circuit is switched onto generate an electrical signal. The circuit of the electromagnetic device 22 in the partition plate 2 is disconnected by the control device 4, so that the attractive force between the movable gasket 3 and the partition plate 2 disappears, and therefore the movable gasket 3 is opened from the fluid drainage outlet 21 to drain the fluid.

In a second embodiment of the control device 4, the center of the elastic closing film 6 is pressed downward along with the rise of the liquid level, so as to drive the movable electrode 42 to move toward the fixed electrode 41 suspended therebelow. Moreover, when the liquid level in the fluid storage cavity 11 reaches the set drainage liquid level, the movable electrode 42 contacts with the fixed electrode 41. At this moment, in the second embodiment the device drains the fluid in away similar to the first embodiment.

The limiting stopper 32 is disposed below the movable gasket 3, and the limiting stopper 32 is configured to enable the electromagnetic device 22 to attract the iron sheet in the movable gasket 3 in a more effective manner, this is beneficial to closing an electronic fluid drainage valve.

In a preferred embodiment of the present invention, the control device 4 further comprises an energy module 43 and a data processing center 45. The energy module 43 is a built-in power source and/or an external power source interface, and the control device 4 further comprises a position judger 46 for judging a displacement status of the drainage container 1.

In a preferred embodiment of the present invention, the control device 4 further comprises an alarm apparatus for indicating the liquid level of the fluid storage cavity.

In a preferred embodiment of the present invention, the control device 4 further comprises a remote control receiving module 44 that is configured to control the movable gasket 3 in a forced manner to open the fluid drainage outlet 21.

The remote control receiving module 44 can receive a signal sent by a remote control unit, and the control device controls the fluid drainage operation in the drainage container based upon the signal received. The remote control unit and the remote control receiving module 44 can be connected in a variety of ways, such as one or more of wireless, infrared, laser and other non-contact communication modes. Besides, the connection between the remote control unit and the remote control receiving module 44 can be in a wired remote control mode.

In a preferred embodiment of the present invention, the remote control receiving module 44 can at least control the drainage container 1 to conduct three procedures, including starting fluid drainage in a forced manner, locking fluid and automatic drainage.

In a preferred embodiment of the present invention, the drainage container 1 is further provided with a data display screen 7, which is configured to display information of the drainage container 1, such as drainage time, total drainage volume and drainage duration.

In a preferred embodiment of the present invention, the drainage container can be equipped with a detachable remote control device, and the remote control device is equipped with three control buttons for starting fluid drainage in a forced manner, locking fluid and automatic drainage. Of course, the remote control device can also be equipped separately.

According to an embodiment of the present invention, the automatic drainage device comprises a drainage container separated into a fluid storage cavity located at an upper portion thereof and a fluid discharge channel located at a lower portion thereof by a partition plate, wherein the fluid storage cavity is provided with a connecting pipe, the fluid discharge channel is provided with a fluid discharge outlet, the partition plate is provided with a fluid drainage outlet that connects the fluid storage cavity to the fluid discharge channel. The device further comprises a movable gasket hinged in the fluid discharge channel located in the lower portion for closing the fluid drainage outlet, a magnetic iron sheet built in the movable gasket, and an electromagnetic device built in the partition plate. The electromagnetic device is operative to attract the magnetic iron sheet to close the fluid drainage outlet. The partition plate is provided with a liquid level monitoring opening. The device further comprises an elastic closing film and a liquid level monitoring unit which are disposed below the liquid level monitoring opening, two fixed electrodes which are vertically disposed in parallel below the elastic closing film, and a limiting stopper disposed below the movable gasket. The device further comprises a control device disposed on a bottom of one side of the partition plate and comprising an energy module and a data processing center, wherein the energy module is a built-in power source. The control device further comprises a spatial position judger for determining a displacement status of the drainage container. The control device further comprises an alarm apparatus for indicating the liquid level of the fluid storage cavity. The control device further comprises a remote control receiving module for forcedly open the movable gasket from the fluid drainage outlet, wherein the remote control receiving module has three procedures, including: starting fluid drainage in a forced manner, locking fluid and automatic drainage. The drainage container is provided with a data display screen.

The drainage fluid flows into the fluid storage cavity located in the upper portion of the drainage container from a drainage container connecting pipe. When the liquid level reaches to a set value, the movable electrode on the elastic closing film is moved into contact with the fixed electrode located therebelow, the contact of the movable electrode and the corresponding fixed electrode switches on the circuit, a signal is generated and sent to the control device. The control device based upon the signal received moves the movable gasket in order to open the fluid drainage outlet. The drainage fluid flows into the fluid discharge channel from the fluid drainage outlet, and finally leaves the device from a fluid discharge outlet.

The present invention is not intended to be limited into the above embodiments. Equal modifications or replacements can further be made by those skilled in the art without departing from the spirit of the present invention and shall all fall within the scope defined by the claims attached.

1. An automatic drainage device, comprising:
a drainage container;
a partition plate, by which the drainage container is separated into a fluid storage cavity located at an upper portion thereof and a fluid discharge channel located at a lower portion thereof, wherein the fluid storage cavity is provided with a connecting pipe, the fluid discharge channel is provided with a fluid discharge outlet, the partition plate is provided with a fluid drainage outlet for connecting the fluid storage cavity and the fluid discharge channel;
a movable gasket, for closing the fluid drainage outlet; and
a control device, comprising a remote control receiving module and configured to controllably force the movable gasket to move for opening the fluid drainage outlet, or the control device is configured to monitor a liquid level in the fluid storage cavity and thereby to controllably force the movable gasket to move for opening and/or closing the fluid drainage outlet.

2. The automatic drainage device according to claim 1, further comprising an electromagnetic device for closing the fluid drainage outlet by means of the movable gasket, wherein the control device comprises at least one liquid level monitoring unit comprising a fixed electrode and a movable electrode, while the liquid level rises to a drainage level, the movable electrode moves into contact with the fixed electrode and a circuit is switched on to generate a signal upon which the control device opens the fluid drainage outlet by means of the movable gasket.

3. The automatic drainage device according to claim 2, wherein the partition plate and the movable gasket are provided with the electromagnetic device and a magnetic iron sheet in a built-in manner, respectively, which are and mutually attracted for closing the fluid drainage outlet, and the electromagnetic device is disposed in the partition plate or the movable gasket.

4. The automatic drainage device according to claim 3, wherein the liquid level monitoring unit further comprises a suspension iron sheet which is fixed by one end thereof, and the movable electrode is fixed at a top end of the suspension iron sheet.

5. The automatic drainage device according to claim 4, wherein the control device is disposed on a side wall of the drainage container, and the control device comprises a liquid level monitoring unit that is flush with a bottom of the fluid storage cavity, and at least one liquid level monitoring unit corresponding to a drainage height.

6. The automatic drainage device according to claim 5, wherein the drainage container is further provided with a vertically-disposed buoy motion rod, and a buoy that can slide up and down along with the buoy motion rod following the liquid level therein, and the buoy is provided with a magnetic material.

7. The automatic drainage device according to claim 6, wherein the control device is disposed at the bottom of the partition plate on one side, the partition plate is provided with a liquid level monitoring opening, an elastic closing film and a liquid level monitoring unit which are disposed below the liquid level monitoring opening, the control device comprises at least two fixed electrodes which are vertically disposed in parallel below the elastic closing film, the movable electrode on the elastic closing film contacts with the fixed electrode located above when the liquid level equals to zero, and the movable electrode on the elastic closing film contacts with the fixed electrode located below when the liquid level is maximum.

8. The automatic drainage device according to claim 1, wherein the movable gasket is by one end thereof hingedly mounted in the fluid discharge channel, and the device further comprises a limiting stopper disposed below a movable end of the movable gasket.

9. The automatic drainage device according to claim 1, wherein the control device further comprises an energy module and a data processing center, and the energy module is a built-in power source and/or an external power source interface.

10. The automatic drainage device according to claim 1, wherein the control device further comprises a space position judger for determining a placement status of the drainage container.

11. The automatic drainage device according to claim 1, wherein the control device further comprises a remote control receiving module, and is configured to forcedly open the fluid drainage outlet by means of the movable gasket.

12. The automatic drainage device according to claim 1, wherein the remote control receiving module controls at least the drainage container to conduct three procedures including forced discharge, locking fluid and automatic drainage.

13. The automatic drainage device according to claim 1, further comprising a data display screen on the drainage container, for displaying data of drainage time, total drainage volume and drainage duration of the drainage container.

14. The automatic drainage device according to claim 1, wherein the fluid drainage outlet is automatically opened when the fluid in the fluid storage cavity is excessive.

15. The automatic drainage device according to claim 1, wherein the partition plate and the movable gasket are provided with the electromagnetic device and a magnetic iron sheet in a built-in manner, respectively, which are and mutually attracted for closing the fluid drainage outlet, and the electromagnetic device is disposed in the partition plate or the movable gasket.

16. The automatic drainage device according to claim 2, wherein the liquid level monitoring unit further comprises a suspension iron sheet which is fixed by one end thereof, and the movable electrode is fixed at a top end of the suspension iron sheet.

17. The automatic drainage device according to claim 1, wherein the control device is disposed on a side wall of the drainage container, and the control device comprises a liquid level monitoring unit that is flush with a bottom of the fluid storage cavity, and at least one liquid level monitoring unit corresponding to a drainage height.

18. The automatic drainage device according to claim 1, wherein the drainage container is further provided with a vertically-disposed buoy motion rod, and a buoy that can slide up and down along with the buoy motion rod following the liquid level therein, and the buoy is provided with a magnetic material.

19. The automatic drainage device according to claim 1, wherein the control device is disposed at the bottom of the partition plate on one side, the partition plate is provided with a liquid level monitoring opening, an elastic closing film and a liquid level monitoring unit which are disposed below the liquid level monitoring opening, the control device comprises at least two fixed electrodes which are vertically disposed in parallel below the elastic closing film, the movable electrode on the elastic closing film contacts with the fixed electrode located above when the liquid level equals to zero, and the movable electrode on the elastic closing film contacts with the fixed electrode located below when the liquid level is maximum.

20. The automatic drainage device according to claim 1, wherein the movable gasket is by one end thereof hingedly mounted in the fluid discharge channel, and the device further comprises a limiting stopper disposed below a movable end of the movable gasket.
